(19) United States
(12) Patent Application Publication
(10) Pub. No.: US 2006/0127996 A1
(43) Pub. Date: Jun. 15, 2006

(54) Method of extraction of isothiocyanates into oil from glucosinolate-containing plants and method of producing products with oil containing isothiocyanates extracted from glucosinolate-containing plants

(75) Inventor: Jed W. Fahey, Baltimore, MD (US)

Correspondence Address: FOLEY AND LARDNER LLP;SUITE 500, 3000 K STREET NW, WASHINGTON, DC, 20007, US

(73) Assignee: Johns Hopkins University

(21) Appl. No.: 11/302,118

(22) Filed: Dec. 14, 2005

Related U.S. Application Data
(60) Provisional application No. 60/635,998, filed on Dec. 14, 2004.

(51) Int. Cl. C12P 13/00

(52) U.S. Cl. 435/128

(57) ABSTRACT

A method of extraction of isothiocyanates into the oil from glucosinolate-containing plants. A method of preparing products including pharmaceutical compositions, food or drink products, supplements or additives, skin or hair products, and agricultural products which contain isothiocyanate oil extracted from glucosinolate-containing plants.

### CROSS-REFERENCE TO RELATED PATENT APPLICATIONS

This application claims priority to U.S. Provisional Application No. 60/635,998 filed Dec. 14, 2004.

### BACKGROUND OF THE INVENTION

1. Field of the Invention

This invention relates to the method for the direct extraction of isothiocyanates into the oil from glucosinolate-containing plants. This invention further relates to the method of preparing products including pharmaceutical compositions, food or drink products, supplements or additives, skin or hair products, and agricultural products which contain the isothiocyanate oil extracted from glucosinolate-containing plants. The administration or ingestion of the products containing the isothiocyanate oil extracted from glucosinolate-containing plants provides a multitude of potential health benefits to the mammals receiving or ingesting said products, including but not limited to, treatment or prevention of allergic response, arterial occlusion, Alzheimer's Disease, cancers (including but not limited to bladder, breast, colon, esophageal, kidney, liver, lung, naso-pharyngeal, ovarian, prostate, skin and stomach), hypo-cholesterolemia, chronic gastritis, hypertension, joint inflammation (arthritis), macular degeneration, stomach ulcers and gastritis, stroke and upper airway inflammation.

2. Background

Plant tissues contain a variety of phytochemicals. Phytochemicals, sometimes referred to as phytonutrients, are naturally occurring and biologically active plant compounds that provide health benefits. Researchers have long known that phytochemicals provide natural health benefits for plants by protecting them against viruses, bacteria fungi, and pests. Certain phytochemicals have been recommended for the purpose of disease prevention and treatment for mammals because of their chemoprotective, antioxidant, and antibiotic properties. Areas of phytochemical research include, but are not limited to, the prevention and treatment of cancer, diabetes, heart disease, hypertension, cataracts, and strengthening of the immune system (Polk, 1996. Feast on Phytochemicals. AICR newsletter. Issue 51) For example, research has revealed that phytochemicals may contribute to cancer chemoprotection, by both reducing the risk of developing several types of cancer and initiating cancer cell apoptosis (Beecher, Am. J. Clin. Nutr., 59(suppl): 1166-70 (1994); Brooks et al., Canc. Epid. Biom. & Prev., September., 10:949-954 (2001); Fahey & Talalay, Phytochemicals and Health, DL Gustine, H E Flores, eds. Rockville, Md.: American Society of Plant Physiologists (1995); Fahey et al., Nutrition Reviews, 57(9) (Part II), September (1999); Fahey et al., Phytochemistry, 56:5-51 (2001); Fahey et al., Proc. Natl. Acad. Sci. USA, May 28;99(11):7610-7615 (2002); Gamet-Payrastre et al. Cancer Research, March 1; 60(5):1426-1433 (2000); Michaud et al., J. Natl. Cancer Inst. 91:605-613 (1999); Prochaska et al., Proc. Natl. Acad. Sci. USA, Mar 15;89(6):2394-8 (1992); Singletary & MacDonald, Cancer Letters, Jul. 3, 155(1):47-54 (2000); Talalay and Fahey, Amer. Soc. Nutr. Sci. (suppl), 3027-3033s. (2001). It is also believed that phytochemicals may be effective for combating or preventing diseases due to their antioxidant effects. The study of phytochemicals for mammal health began in earnest, when research began identifying a connection between phytochemicals containing chemoprotective and antioxidants properties and cellular protection, repair and regeneration (T. J. Smith, Renewal: The Anti-Aging Revolution. (1998)).

One of the classes of phytochemicals is called glucosinolates, and glucosinolates are also present in a wide variety of plants. Over 500 plant species contain glucosinolates, of which 16 glucosinolate families are known. Examples of plants that contain glucosinolates or isothiocyanates include, but are not limited to, Brassicacae (Cruciferae), Moringaceae and Resedaceae, which collectively include, but are not limited to, broccoli, broccoli sprouts, Brussels sprouts, cabbage, cauliflower, cauliflower sprouts, daikon, horseradish, kale, mustard seed, radish, wasabi, horseradish tree (Moringa oleifera), cabbage tree (M. stenopetala), mignonette (Reseda odorata), dyer's rocket (R. luteola). Other families of plants that contain glucosinolates include, but are not limited to, Bataceae, Bretschneideraceae, Capparaceae, Caricaceae, Euphorbiaceae, Gyrostemonaceae, Limnanthaceae, Pentadiplandraceae, Phytolaccaceae, Pittosporaceae, Salvadoraceae, Tovariaceae and Tropaeolaceae (and these include plants such as capers (Capparis spinosa), and nasturtium (Tropaeolum majus)). The high levels of glucosinolates may occur naturally in plants or plants may be bred to contain higher levels or glucosinolates.

For example, glucosinolates, which are converted by enzymatic hydrolysis to isothiocyanates, have been identified as having anti-cancer potential. ((Fahey et al., Proc. Natl. Acad. Sci USA, 94: 10367-10372 (1997) Posner et al., J. Med. Chem., 17:170-175(1994), Shapiro et al., Canc. Epid. Biom. & Prev., 7(12): 1091-1100 (1998), Talalay et al., J. Nutrition, 131:3027S-3033S (2001), Zhang et al., Proc. Natl. Acad. Sci. USA, 89: 2399-2403 (1992), and Zhang et al., Proc. Natl. Acad. Sci. USA, Apr. 12, 91(8):3147-50 (1994)). In addition, isothiocyanates have great value as antibiotics (Fahey et al., Proc. Natl. Acad. Sci USA, 99:7610-7615 (2002) and U.S. Pat. No. 6,737,441), and for their utility in protection of mammals against chronic disorders associated with aging and oxidative stress (Dinkova-Kostova et al., Proc. Natl. Acad. Sci. USA, 102:4584-4589 (2005), Fahey and Talalay, Food Chem. Toxicol., 37:973-79 (1999); Fahey et al., Phytochemistry, 56:5-51 (2001), Fahey et al. Carcinogenesis, July, 26(7):1247-55 (2005), Gao et al., Proc. Natl. Acad. Sci. USA, December 18; 98(26): 15221-15226 (2001), Healy et al., Proc. Nat. Acad. Sci. USA, September, 102(39):10410-10415 (2005), Holtzclaw et al., Adv Enzyme Regul., 44:335-67 (2004), Talalay and Dinkova-Kostova, Methods Enzymol., 382:355-64 (2004) and Talalay et al., Adv Enzyme Regul, 43:121-34 (2003)). Further, isothiocyanates possess agricultural benefits, due to their fungicidal, bactericidal, and insect repellent properties. Specifically, isothiocyanates are biologically active and possess the foregoing benefits through their ability to induce Phase 2 detoxification enzymes in mammals.

Highly efficient methods have been developed for measuring the potency of plant extracts to increase or induce the activities of Phase 2 enzymes. (Prochaska et al., Anal. Biochem. 169: 328-336 (1988) and Prochaska et al., 1992). In addition, these methods have been employed for isolating glucosinolates and their cognate isothiocyanates responsible for the inducer activities in plants and for evaluating the potential health benefits these compounds provide to mammals ingesting or being administered the isolated isothiocyanate. (Zhang et al., Proc. Natl. Acad. Sci. USA, 89: 2399-2403 (1992) and Posner et al., J. Med. Chem., 17: 170-175 (1994)).

Glucosinolates are water-soluble, stable compounds, which are converted to isothiocyanates by the enzyme, myrosinase, which co-exists within the plant tissues. Isothiocyanates are much less water soluble and more highly fat soluble (lipophilic). The myrosinase enzymatic reaction (conversion of glucosinolate precursors to isothiocyanates) occurs only in hydrated conditions, and this reaction produces a less water soluble and biologically active compound from one that is highly water soluble, and relatively non-reactive.

In this invention, methods are described for the simultaneous conversion of glucosinolates to their cognate isothiocyanates from plant material, partition into the plant material oil, and extraction from the plant material. The plant material is moistened, homogenized, incubated for a number of hours to allow complete enzymatic conversion of glucosinolates to isothiocyanates, and then the oil is removed from the plant material. Oil removal can be done via a number of methods such as cold-pressing (screw-press), solvent extraction, supercritical CO₂ extraction, fractional distillation, hydraulic pressing, and expeller pressing. Pre-treatment may be required for certain plant parts (e.g. seeds), such as dehulling, breaking, grinding, flaking, rolling, pressing, and pelleting, and thermal treatment may be required to degrade cell walls and to reduce oil viscocity (Intermediate Technology Development Group, Principles of Oil Extraction: A Technical Brief, ITDG Publishing, pp. 1-11).

Embodiments of this invention include the addition of ascorbate to the enzymatic digest to facilitate complete hydrolysis, inactivation of epithiospecifier protein in species where it occurs, and the use of ethyl acetate as a solvent for oil extraction.

Another embodiment of this invention is the simultaneous conversion of glucoraphanin, to its cognate isothiocyanate, sulforaphane, from plant material, partition into the plant material oil, and extraction from the plant material. Stabilization of the isothiocyanates accompanies transfer to the oil phase. When in the aqueous phase, isothiocyanates can react with proteins, whereas in the oil phase they are essentially isolated from this substrate. Thus stabilization accompanies partition and it is precisely this in-situ, rapid, transfer into the oil phase which confers stability to the isothiocyanates.

Glucoraphanin is one of the most abundant glucosinolates in broccoli. Its cognate isothiocyanate sulforaphane, is a potent inducer of mammalian detoxification and chemoprotection by inducing Phase 2 enzymatic activity. (U.S. Pat. Nos. 5,725,895; 5,968,505; 5,968,567 and 6,521,818; and Zhang et al., Proc. Natl. Acad. Sci. USA, 89:2399-403 (1992)).

Another embodiment of this invention is the simultaneous conversion of glucoraphanin, to its cognate isothiocyanate, sulforaphane, from broccoli plant seeds, partition into the seed oil, and extraction from the seed.

Thus, the present invention transfers the isothiocyanate into the natural oil already present in plant materials. This method confers stability to the isothiocyanate, thereby providing a new and effective vehicle for administering the natural, oil-based isothiocyanate formulation to mammals and plants. The combined stability and non-aqueous properties of the extracted isothiocyanate oil expands the potential for different types of applications, including but not limited to, dermal absorption, inhalation (nasal sprays), ingestion, injection, and spray application. Further, the natural, oil-based delivery vehicle enhances the potential commercial use of the isothiocyanate oil extract either alone or as a component in the compositions of pharmaceutical products, food or drink products, supplements and additives, skin or hair products, and agricultural products.

### SUMMARY OF THE INVENTION

The present invention provides for the method of extracting isothiocyanates from glucosinolate-containing plant material comprising a) crushing glucosinolate-containing plant material; b) moistening the crushed glucosinolate-containing plant material; c) incubating the moistened and crushed glucosinolate-containing plant material to allow for conversion of glucosinolates to isothiocyanates; and d) removing oil containing isothiocyanates from the material from part c).

Another aspect of the present invention provides for the above method wherein said plant material is crushed by any of a variety of crushing or grinding or flaking devices common to the seed and grain industry, including but not limited to, dehulling, breaking, grinding, flaking, rolling, pressing, and pelleting.

Another aspect of the present invention provides for the above method wherein said plant material is subjected to thermal treatment to degrade cell walls and to reduce oil viscosity.

Another aspect of the present invention provides for the above method wherein said plant material is moistened with a minimal amount of water, wherein the amount of water needed is adjusted based upon the type of plant material, and is calibrated to moisten, without resulting in an overabundance of standing or free water, in order that the myrosinase enzyme contained within plant tissues can become fully operational.

Another aspect of the present invention provides for the above method wherein said plant material is pre-incubated at a temperature of about 60° C. for the length duration within the range of about 5 minutes to 3 hours to maximize the conversion of glucosinolate to isothiocyanate whilst inactivating the epithiospecifier protein if it exists in that plant material (not all plants contain this protein).

Another aspect of the present invention provides for the above method wherein step c) further comprises the addition of ascorbate, myrosinase, both ascorbate and myrosinase, or any enzyme which facilitates the conversion of isothiocyanate to glucosinolate.

Another aspect of the present invention provides for the above method wherein said oil removal is by a method selected from the group consisting of cold press extraction (screw-press), solvent extraction, fractional distillation, hydraulic pressing, expeller pressing and supercritical CO₂ extraction.

Another aspect of the present invention provides for the above method wherein said extraction with solvents is performed with one or more solvents selected from the group consisting of, but not limited to hexane, methylene chloride, and ethyl acetate.

Another aspect of the present invention provides for the above method wherein said solvent is ethyl acetate.

Another aspect of the present invention provides for the above method wherein said solvent is hexane.

Another aspect of the present invention provides for the above method wherein said isothiocyanate is one or more of sulforaphane, sulforaphene, erysolin, erucin, iberin, alyssin, berteroin, iberverin, cheirolin, 5-methylsulfinylpentyl isothiocyanate, 6-methylsulfinylhexyl isothiocyanate, 7-methylsulfinylheptyl isothiocyanate, 8-methylsulfinyloctyl isothiocyanate (hirsutin), 9-methylsulfinylnonyl isothiocyanate, 10-methylsulfinyldecyl isothiocyanate, phenylethyl isothiocyanate, 4-(α.-L-rhamnopyranosyloxy)benzyl isothiocyanate, 3-(α.-L-rhamnopyranosyloxy)benzyl isothiocyanate, 2-(α L-rhamnopyranosyloxy)benzyl isothiocyanate, 4-(4′-O-acetyl-alpha.-L-rhamnopyranosyloxy)benzyl isothiocyanate or a derivative thereof.

Another aspect of the present invention provides for the above method wherein said isothiocyanate is sulforaphane.

Another aspect of the present invention provides for the oil made by the above method.

Another aspect of the present invention provides for the pharmaceutical composition comprising the oil produced by the above method and a pharmaceutically acceptable excipient, wherein said pharmaceutical composition is selected from the group consisting of, but not limited to, antibiotic, antifungal, antihistamine, anti-hypertension, anti-protzoal, antifilarial, anti-malarial, anti-schistosomal, anti-ulcer, anti-coagulant, anti-anxiety, anti-inflammation, antiseptic, nematocidal, antiviral, vasodilators, and protective/prophylactic, and can be administered orally, nasally, parenterally, intrasystemically, intraperitoneally, topically (as by drops of transdermal patch), bucally, or as an oral or nasal spray, and wherein said pharmaceutical composition can be used for human or veterinary applications.

Another aspect of the present invention provides for the food or drink product, supplement or additive comprising the oil produced by the above method, wherein said food or drink product or supplement is selected from the group consisting ofjuices, smoothies, shakes, teas, soups, sauces, salads, granolas, cereals, breads, other baked goods, fried goods, pills, sprays and other ingestible products, and wherein said food or drink product, supplement or additive can be used for human or veterinary applications.

Another aspect of the present invention provides for the skin or hair product comprising the oil produced by the above method, wherein said skin or hair product is selected from the group consisting of hair detergents such as shampoo, rinse, rinse-in-shampoo, conditioning shampoo, and the like; various hair cosmetics including hair lotion, hair conditioner, hair treatment, hair cream, hair spray, hair liquid, hair wax, hair water, hair-styling preparation, penning liquid, hair color, acidic hair color, hair manicure, etc.; or various skin cosmetics such as skin lotion, milky lotion, face wash, makeup remover, cleansing lotion, emollient lotion, nourishing cream, emollient cream, massage cream, cleansing cream, body shampoo, hand soap, bar soap, shaving cream, sunburn cosmetics, deodorant gel, deodorant powder, deodorant lotion, deodorant spray, anti-perspirant gel, antiperspirant powder, anti-perspirant lotion, anti-perspirant spray, combination deodorant & anti-perspirant gel, combination deodorant/anti-perspirant powder, combination deodorant/anti-perspirant lotion, combination deodorant/anti-perspirant spray, makeup removing gel, moisture gel, moisture essence, UV-preventing essence, shaving foam, face powder, foundation, lipstick, cheek rouge, eyeliner, eye shadow, eyebrow pencil, bathing preparation, etc.; mouth detergent such as toothpaste; and other hair and skin products, and wherein said skin or hair product can be used for human or veterinary applications.

Another aspect of the present invention provides for the agricultural product comprising the oil produced by the above method, wherein said agricultural product is selected from the group consisting of agricultural pesticides, powders, pellets, sprays, fertilizers, side-dressings, in-furrow amendments, soil amendments, composts and other agricultural products.

The present invention further provides for the method of inducing the activity of phase 2 enzymes in a mammal comprising administering an effective amount of the above pharmaceutical composition to a mammal in need thereof.

The present invention further provides for the method of inducing the activity of phase 2 enzymes in a mammal comprising administering an effective amount of the above food or drink product, supplement or additive to a mammal in need thereof.

The present invention further provides for the method of inducing the activity of phase 2 enzymes in a mammal comprising administering an effective amount of the above skin or hair product to a mammal in need thereof.

The present invention further provides for the method of inducing the activity of phase 2 enzymes in a plant comprising administering an effective amount of the above agricultural product of claim 23 to a plant in need thereof.

The present invention further provides for the method of treating or preventing skin cancer in a mammal comprising administering the above skin product to the skin of said mammal.

The present invention further provides for the method of treating or preventing allergic response, arterial occlusion, Alzheimer's Disease, cancer, hypo-cholesterolemia, chronic gastritis, hypertension, joint inflammation (arthritis), macular degeneration, stomach ulcers and gastritis, stroke and upper airway in a mammal comprising administering an effective amount of the above pharmaceutical composition to said mammal.

The present further provides for the method of making a product containing isothiocyanates comprising a) crushing glucosinolate-containing plant material; b) moistening the crushed glucosinolate-containing plant material; c) incubating the moistened crushed glucosinolate-containing plant material to allow for conversion of glucosinolates to isothiocyanates; d) removing oil containing isothiocyanates from the material from part c); and e) adding the oil from part (d) to a pharmaceutical product, a food or drink product, supplement or additive, a skin or hair product, or an agricultural product.

### BRIEF DESCRIPTION OF THE DRAWINGS

N/A

### DETAILED DESCRIPTION OF THE INVENTION

All references cited herein are incorporated in their entirety by reference.

1. Definitions

In the description and tables which follow, a number of terms are used. In order to provide a clear and consistent understanding of the present invention, the following definitions are provided:

A chemoprotector or chemoprotectant is a synthetic or naturally occurring chemical agent that reduces susceptibility in a mammal to the toxic and neoplastic effects of carcinogens.

A phytochemical is a naturally occurring, non-nutritive plant chemical that has protective or disease preventive properties to mammals. Phytochemicals perform some of the following biochemical processes: antioxidant activity, hormonal, stimulation of different enzymes, interference with DNA replication, and antibacterial or antifungal effect.

A monofunctional inducer increases the activity of Phase 2 enzymes selectively without significantly altering Phase 1 enzyme activities. Monofunctional inducers do not depend on a functional Ah receptor but enhance transcription of Phase 2 enzymes by means of an Antioxidant Responsive Element (ARE). Sulforaphane is a monofunctional inducer.

A bifunctional inducer increases 1) activities in both Phase 1 enzymes, such as ctyochromes P-450, and Phase 2 enzymes, and 2) requires the participation of an Aryl hydrocarbon (Ah) receptor and its cognate Xenobiotic Response Element (XRE). Examples include flat planar aromatic such as polycyclic hydrocarbons, azo dyes, or 2,3,7,8-tetrachloro-dibenzo-p-dioxin (TCDD).

Glucosinolates, which are well known in the art, and are phytochemicals which occur in all plant tissues and degrade via enzymatic hydrolysis. Glucosinolates are grouped as either aliphatic, aromatic, or indole forms. Enzymatic hydrolysis of glucosinolates yields nitriles, epithionitriles, thiocyanates, and/or isothiocyanates depending on the parent glucosinolate, pH and other factors. Examples of glucosinolates include, but are not limited to, glucoraphanin, glucoerysolin, glucoerucin, glucoiberin, glucoalyssin, glucoberteroin, glucoiberverin, glucocheirolin, glucoraphenin, 5-methylsulfinylpentyl glucosinolate, 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyldecyl glucosinolate, phenylethyl glucosinolate, 4-(α-L-rhamopyranosyloxy)benzyl glucosinolate, 3-(α-L-rhamnopyranosyloxy)benzyl glucosinolate, 2-(α-L-rhamnopyranosyloxy)benzyl glucosinolate, 4-(4′-O-acetyl-α-L-rhamnopyranosyloxy)benzyl glucosinolate as well as those reviewed in Table 1 of Fahey et al., Phytochemistry, 56:5-51 (2001).

Isothiocyanates are released through enzymatic hydrolysis of glucosinolates by myrosinase. Isothiocyanates are compounds containing the thiocyanate (SCN) moiety and are easily identifiable by one of ordinary skill in the art. The description and preparation of isothiocyanate analogs is described in U.S. Reissue Pat. No. 36,784, and is hereby incorporated by reference in its entirety. An example of an isothiocyanate includes, but is not limited to, sulforaphane (4-methylsulfinylbutyl isothiocyanate or (−)-1-isothiocyanato-4(R)-(methylsulfinyl) butane) or its analogs.

An epithiospecifier protein (ESP) is a protein that catalyses formation of nitriles or epithionitriles during glucosinolate hydrolysis by myrosinase. After myrosinase hydrolysis, epithionitriles can be generated by the ESP protein in the presence of iron and a favorable pH; however, in the absence of ESP, glucosinolates convert to isothiocyanates. Heating of a plant material, such as broccoli, for 10 minutes at 140° F., kills the ESP protein while not affecting the enzymatic activity of myrosinase, in turn, maximizing the conversion of glucosinolate to its cognate isothiocyanate (Jeffrey et al. Maximizing the Anti-Cancer Power of Broccoli. Science Daily, p. 1, (2005), Kliebenstein et al. Current Opinion in Plant Biology, 8:264-271 (2005) and Matusheski et al., Phytochemistry, May, 65(9):1273-81 (2004).

An electrophile is a molecule that has a positively charged center, so that it can react with electron-rich centers such as those that exist in DNA and cause damage. Many cancer-causing chemicals are electrophiles or converted to electrophiles.

Glutathione (GSH) is a naturally occurring peptide, serving as a biological redox agent or a coenzyme, present in very high concentrations in cells. It is the principal protective natural antioxidant that protects cells against oxidative damage.

A glucosinolate concentration is the average amount of glucosinolate produced per gram of selected plant material.

Inducer activity or Phase 2 enzyme-inducing activity is a measure of the ability of a compound(s) to induce Phase 2 enzyme activity. (Prochaska et al., Anal. Biolchem., 169:328-336 (1988); Prochaska et al., 1992; Fahey 2004 Methods in Enzymology).

Inducer potential or Phase 2 enzyme-inducing potential is a measure of the combined amounts of inducer activity in plant tissue provided by isothiocyanates, plus glucosinolates that can be converted by myrosinase to isothiocyanates. Glucosinolates are not themselves direct inducers of mammalian Phase 2 enzymes; instead, their metabolic products, isothiocyanates, are inducers. The inducer potential, as distinct from inducer activity, of plant extracts can be measured by adding purified myrosinase, obtained from the same, or other plant sources, to an assay system. Inducer potential can be measured using a multiwell plate screen with murine hepatoma cells for in vitro measurement of QR specific activity.

Plant material is defined as plant tissue, whole plants, and plant parts consisting of seeds, fruit, sprouts, leaves, stems, tubers, flowers and roots.

A mammal is defined as an endothermic or “warm-blooded” vertebrate animal that has the presence of 1) mammary glands, which in the females produces milk for the nourishment of the young, and 2) hair or fur. The mammal class includes about 5500 species, 1200 genera, 152 families and 46 orders. The class of mammals includes human beings, apes, many four-legged animals, whales, dolphins, and bats.

A leachate is defined as a liquid containing soluble material which was removed from a solid mixture through which liquid was passed.

Hydrolysis is a chemical reaction in which a compound reacts with water, causing decomposition and results in the production of two or more compounds.

Enzymatic hydrolysis is a chemical reaction in which a compound reacts with water and an enzyme, causing decomposition and results in the production of two or more compounds. In this invention, glucosinolate is converted to its cognate isothiocyanate by the enzyme, myrosinase.

A vehicle, in the pharmaceutical industry, is defined as an inactive substance blended with a drug or active substance which enables for easier application, ingestion of administering of the active substance.

A solvent extraction is the technique for removing the desired component by transferring the compound from an aqueous to an organic solvent that can be separated.

A pharmaceutical product is any preparation containing an isothiocyanate extracted into oil from a glucosinolate-containing plant which is capable of delivering that isothiocyanate to the mammal administered the pharmaceutical product. The pharmaceutical product can be an antibiotic, antifungal, antihistamine, anti-hypertension, anti-protzoal, antifilarial, anti-malarial, anti-schistosomal, anti-ulcer, anti-coagulant, anti-anxiety, anti-inflammation, antiseptic, vasodilator, protective/prophylactic, or other pharmaceutical product. The pharmaceutical product can be administered orally, nasally, parenterally, intrasystemically, intraperitoneally, topically (as by drops of transdermal patch), bucally, or as an oral or nasal spray. The term “parenterally” refers to the modes of administration which include intravenous, intramuscular, intraperitoneal, intrastemal, subcutaneous and intraarticular injection and infusion. Solid dosage forms of the pharmaceutical product include, but are no limited to, capsules, dragees, tablets, pills, powders and granules. Liquid dosage forms of the pharmaceutical product include, but are not limited to pharmaceutically acceptable emulsions, solutions, suspensions, syrups and elixirs. In this invention, the pharmaceutical product can be used for either human or veterinary applications.

A food or drink product, supplement or additive is any ingestible preparation containing an isothiocyanate extracted into oil from a glucosinolate-containing plant which is capable of delivering that isothiocyanate to the mammal ingesting the food or drink product, supplement or additive, from the group consisting of, but not limited to, juices, smoothies, shakes, teas, soups, sauces, salads, granolas, cereals, breads, other baked goods, fried goods, pills, sprays or other ingestible products, supplements or additives. The dose of isothiocyanate added to a food or drink product, supplement or additive preferably is in the range of 1 μmol to 1,000 μmol per serving. However, the dose of glucosinolate and/or isothiocyanate supplementing the food product can be higher. In this invention, the food or drink product, supplement or additive can be used for either human or veterinary applications.

A skin or hair product is any dermal preparation containing an isothiocyanate extracted into oil from a glucosinolate-containing plant which is capable of delivering those isothiocyanates to the mammal being administered the skin or hair product. The skin or hair product can be hair detergents such as shampoo, rinse, rinse-in-shampoo, conditioning shampoo, and the like; various hair cosmetics including hair lotion, hair conditioner, hair treatment, hair cream, hair spray, hair liquid, hair wax, hair water, hair-styling preparation, perming liquid, hair color, acidic hair color, hair manicure, etc.; or various skin cosmetics such as skin lotion, milky lotion, face wash, makeup remover, cleansing lotion, emollient lotion, nourishing cream, emollient cream, massage cream, cleansing cream, body shampoo, hand soap, bar soap, shaving cream, sunburn cosmetics, deodorant gel, deodorant powder, deodorant lotion, deodorant spray, anti-perspirant gel, anti-perspirant powder, anti-perspirant lotion, anti-perspirant spray, combination deodorant & anti-perspirant gel, combination deodorant/anti-perspirant powder, combination deodorant/anti-perspirant lotion, combination deodorant/anti-perspirant spray, makeup removing gel, moisture gel, moisture essence, UV-preventing essence, shaving foam, face powder, foundation, lipstick, cheek rouge, eyeliner, eye shadow, eyebrow pencil, bathing preparation, etc.; mouth detergent such as toothpaste; or other skin and hair products. In this invention, the skin or hair product can be used for either human or veterinary applications.

An agricultural product is any preparation containing an isothiocyanate extracted into oil from a glucosinolate-containing plant which is capable of delivering those isothiocyanates to the agricultural system being treated. The agricultural product can be agricultural pesticides, powders, pellets, sprays, fertilizers, composts, soil amendments, in-furrow applications, or other agricultural products.

2. Glucosinolates Acting as Chemoprotectants

One of the body's first lines of defense against cancer is Phase 2 enzymes. Phase 2 enzymes are central to the body's ability to protect itself from all carcinogens that routinely enter through the diet and the environment. The fact that Phase 2 enzymes can defend against so many pre-carcinogenic or carcinogenic compounds is what makes them a major research focus in the prevention and treatment of cancer.

Mammalian cells contain Phase 1 and Phase 2 enzymes. Phase 1 enzymes frequently convert pre-carcinogenic compounds that have entered the cell through diet or the environment and make them more reactive, more water-soluble and easier for the body to dispose of (often via the action of Phase 2 enzymes). When Phase 2 enzymes are induced by certain compounds, the cell becomes more able to detoxify the carcinogens (some produced by the action of Phase 1 enzymes), which would otherwise damage DNA an initiate cancer development. Phase 2 enzymes can attack carcinogens directly, render them inert, or facilitate their excretion from the cell.

It is now believed that a major mechanism of chemoprotective protection and treatment depends of the presence of chemical compounds in plants that, when administered to mammalian cells, elevate levels of Phase 2 enzymes that detoxify carcinogens. Compounds which elevate the level of Phase 2 enzymes are termed “selective inducers.” Selective inducers of Phase 2 enzymes are designated monofunctional inducers. This means that they induce only Phase 2 enzymes, without significantly inducing Phase 1 enzyme activities. In contrast, compounds which induce both Phase 2 and Phase 1 enzymes are designated bifunctional inducers. (Prochaska et al. Cancer Research 48:4776-4782 (1988)). The monofunctional inducers are nearly all electrophiles and belong to at least 10 distinct chemical classes. (Dinkova-Kostova et al. Canc. Epid. Biom. & Prev., November, 14(11), Fahey et al. Carcinogenesis, July, 26(7):1247-55 (2005), Prestera et al., Proc. Natl. Acad. Sci. USA, 90: 2963-2969 (1993) and Khachick et al., In Antioxidant Food Supplements in Human Health, Packer, L. et al. (eds), San Diego: Academic Press, pp. 203-229 (1999).

Monofunctional inducers are chemoprotective agents which reduce the susceptibility of mammals to the toxic and neoplastic effects of carcinogens Chemoprotectors can be of plant origin or synthetic compounds. Synthetic analogs of naturally occurring inducers have also been generated and shown to block chemical carcinogenesis in animals. (Posner et al., 1994; Zhang et al., Proc. Natl. Acad. Sci. USA, 91: 3147-50 (1994); and Zhang et al., Cancer Research, (Suppl) 54: 1976s-1981s (1994)).

It is now known that most of the inducer activity of crucifer plants is due to the presence and amounts of isothiocyanates and their biogenic precursors, glucosinolates. Glucosinolates are converted to isothiocyanates by the enzyme myrosinase, which is a thioglucoside glucohydrolase. Normally, myrosinase and glucosinolates are separated in the cell. If the cell is damaged, resulting in disruption of cellular compartmentalization, myrosinase comes into contact with glucosinolates, and converts them to isothiocyanates. Although glucosinolates are not themselves inducers of mammalian Phase 2 enzymes, their conversion products, by virtue of myrosinase activity, are. Thus, it is the isothiocyanate products which are potent monofunctional inducers of Phase 2 enzymes.

However, not all glucosinolates produce isothiocyanates which are inducers of Phase 2 enzymes. Certain glucosinolates (e.g. alkylthioalkyl glucosinolates) produce isothiocyanates that are potent chemoprotective agents. Other glucosinolates (e.g. indole glucosinolates) produce compounds, such as indole-3-carbinol and indole-3-acetonitrile, that are problematic for several reasons. First, such indole glucosinolates are bifunctional inducers; that is, they induce both Phase 1 and Phase 2 enzymes. Phase 1 enzymes can activate xenobiotics thereby creating carcinogens. (Prochaska & Talalay, Cancer Research, 48: 4776-4782 (1988)). Second, the indole glucosinolates are only weak inducers of Phase 2 enzymes (Fahey et al., Chapter 2 in Functional Foods for Disease Prevention, I. Shibamoto T. et al. (eds), ACS Symposium Series 701, Washington D.C.: Am. Chem. Soc., pp. 16-22 (1998)). Third, these compounds themselves function as tumor promoters (Kim et al., Carcinogenesis, 18(2):377-381 (1997)). Finally, these compounds can form condensation products under the acid conditions encountered in the stomach, which are potent carcinogens very similar to dioxin (TCDD) (Bjeldanes et al., Proc. Nat. Acad. Sci. USA, 88:9543-9547 (1991)).

Thus, the amount of Phase 2 inducer activity depends upon both the quality and quantity of glucosinolates and their cognate isothiocyanates. Scientific research has shown that certain foods, like crucifers such as broccoli, contain natural glucosinolate compounds which are able to selectively boost only Phase 2 enzymes.

3. Glucosinolates Acting as an Indirect Antioxidant

Direct antioxidants neutralize free radicals before they can harm cells. Vitamins C and E and beta-carotene are direct antioxidants. Indirect antioxidants do not neutralize free radicals directly, but rather boost Phase 2 enzymes that trigger ongoing and long-lasting antioxidant activity. Isothiocyanates are indirect antioxidants. For example, researchers believe that the isothiocyanate, sulforaphane, may be even more effective than direct antioxidants in protecting cells against free radicals and electrophiles (Fahey and Talalay, Food Chem Toxicol., 37:973-79 (1999) and Khachick et al. In Antioxidant Food Supplements in Human Health, pp. 203-229 (1999)).

4. Glucosinolates and Isothiocyanates Agricultural Uses

The biological effects of glucosinolates, and their cognate isothiocyanates, in agriculture have been researched extensively and are well summarized by Fahey et al., Phytohemistry, 56:5-51 (2001) and by others. Glucosinolates, and their cognate isothiocyanates, have been utilized in agriculture for their property to act as: an antibacterial, antibiotic, antimicrobial, antifungal, antiprotozoal, nematocidal and insect repellent.

5. Suloraphane as Chemoprotectant, Antioxidant and Antibiotic

The isothiocyanate, sulforaphane, found in cruciferous vegetables, and particularly broccoli, has been researched as a phytochemical with very promising disease prevention and treatment properties. Sulforaphane was first isolated and identified in 1992 (Zhang et al., Proc. Natl. Acad. Sci. USA, 89:2399-2403 (1992)). Sulforaphane is one of the most potent, naturally occurring inducers of Phase 2 enzymes known to date, which thus, makes this isothiocyanate a natural anticancer agent. (U.S. Pat. Nos. 5,411,986; Re. 36,784; 5,725,895; 5,968,505; 5,968,567; 6,177,122; 6,242,018; 6,521,818; 6,737,441 and PCT Patent WO 97/09889) Published research findings have also shown that sulforaphane stimulates apoptosis and inhibits proliferation (Gamet-Payrastre et al., Cancer Res., 60:1426-1433 (2000)), is anti-inflammatory (Heiss et al., J. Biol. Chem., 276:32008-32015 (2001)), inhibits histone deacetylase (Myzak et al., Cancer Res., 64:5767-5774 (2004)), and sulforaphane and a small number of other isothiocyanates are directly and selectively antibacterial against Helicobacter pylori, a widespread human pathogen (Fahey et al., Proc. Natl. Acad. Sci. USA, May 28, 99(11): 7610-15 (2002) and Haristoy et al., Planta Medica, 71: 326-330 (2005).

In addition, new research findings continue to be published revealing the expanding range of potential health benefits sulforaphane offers for the prevention and treatment of a multitude of diseases and disorders, including but are not limited to 1) allergic response, 2) arterial occlusion, 2) Alzheimer's Disease, 3) cancers (including but not limited to bladder, breast (Singletary et al., Cancer Letters, 155(1):47-54 (2000)), colon (Chung et al., Proc. of the Amer. Assoc. Canc. Res., 41:660 (2000); Chung et al., Carcinogenesis, 21, 2287-2291 (2000) and (Garnet-Payrastre et al., Cancer Research, 60(5):1426-1433 (2001)), esophageal, kidney, liver, lung (Conway et al. Can. Res., 65(18): 8548-8557 (2005) and Kensler et al., Canc. Epid. Biom. & Prev., November 2005, 14(11):2605-2613), naso-pharyngeal, ovarian, prostate Brooks et al, Canc. Epid., Biom. & Prev., 10:949-954 (2001)), skin (Dinkova-Kostova et al., Canc. Epid. Biom. & Prev., November 2005, 14(11)(Part II):2690s and Dinkova-Kostova et al., Cancer Letters, e-pub Nov. 2, 2005, and stomach (Yanaka et al., Canc. Epid. Biom. & Prev., November, 14(11)(Part II):2754s (2005); Fahey et al., Proc. Natl. Acad. Sci. USA, May 28, 99(11): 7610-15 (2002) and Galan et al., Dig Dis Sci., 49(7-8):1088-90 (2004)₄) hypo-cholesterolemia (Murashima et al. BioFactors, 22:271-275 (2004))., 5) chronic gastritis, 6) hypertension (Wu et al., Hypertension, 19:1819-1825 (2001) and Wu et al., Proc. Natl. Acad. Sci. USA, 101(18): 7094-7099 (2004)), 7) joint inflammation (arthritis) (JHMI Press Release: Phytochemicals May Protect Cartilage, Prevent Paid in Joints: Phase 2 Enzyme Inducers Appear to Stop Harmful Inflammation and Healy et al., Proc. Nat. Acad. Sci. USA, 102(39):10410-10415 (2005)), 8) macular degeneration (Gao et al., Proc. Natl. Acad. Sci. USA, 98(26):15221-15226 2001); (Gao et al., Proc. Natl. Acad. Sci. USA, 101: 10446-1045 (2004))., 9) stomach ulcers and gastritis(Fahey et al., Proc. Natl. Acad. Sci. USA, 99(11):7610-7615. 15 (2002). stroke (Zhao et al. Neurosci. Lett., e-pub Oct. 15, 2005 and upper airway inflammation.

6. Direct Extraction of Glucosinolates from Plant Material

If fresh-picked vegetables are promptly and gently harvested, directly into organic solvents, comprising a mixture of DMF/ACN/DMSO/H₂O and a temperature that prevents myrosinase activity, both glucosinolates and isothiocyanates are efficiently extracted into the organic solvent mixture. Preferably, the DMF, ACN, DMSO, and H₂O are mixed in equal volumes. However, the volumes of the three solvents in the mixture can be varied to optimize extraction of specific glucosinolates and isothiocyanates from any plant tissue. The temperature of the extraction mixture is preferably less than 0° C. The temperature of the extraction solvent must be kept above its freezing point. At the same time the enzyme myrosinase, which invariably accompanies these constituents in the plants and rapidly converts glucosinolates into isothiocyanates, is inactive. Such extracts typically contain high quantities of glucosinolates and negligible quantities of isothiocyanates. The in planta myrosinase activity varies between different plant species.

Glucosinolates are converted at least partially to isothiocyanates in mammals. If, however, it is desirable to accelerate this conversion, broccoli or other vegetable sprouts, high in glucosinolates, can be mixed with myrosinase. The mixture can be in water, or some other non-toxic solvent that does not inactivate myrosinase. The myrosinase can be from a partially purified or purified preparation. Alternatively, the myrosinase can be present in plant tissue, such as a small quantity of crucifer sprouts rich in myrosinase.

Direct extraction of the isothiocyanates from plant tissues into a stable and instantly usable non-aqueous form has been problematic. This invention provides a description of the direct extraction of isothiocyanates from plant materials into natural plant oils. This invention also identifies methods for simultaneous myrosinase-catalyzed conversion of a glucosinolate, to its active metabolite, an isothiocyanate, and extraction of that active metabolite. The invention further describes the simultaneous production and extraction of isothiocyanates as a stable and natural, oil-based delivery vehicle which can be incorporated into many different commercial products such as pharmaceuticals products, food or drink products, supplements and additives, skin or hair products and agricultural products.

Direct extraction is a process wherein extraction and hydrolysis are completed in one step. The benefits of direct extraction include reduction in processing time and cost, as well as, improved quality of the extract since all of the purification steps are performed after hydrolysis. The active ingredient (the isothiocyanate) is stable, due to a lack of reaction (self-destruction), with its preferred substrate (e.g. plant proteins). Due to the nature of the process in which water soluble glucosinolates are converted to isothiocyanates in an aqueous milieu, and then can rapidly be removed from the presence of particulate contaminants and concentrated in the lipid (oil) phase of the plant which is then extracted. Types of direct extraction include: cold press, hydraulic pressing, expeller pressing, fractional distillation, solvent extraction, and supercritical CO₂ extraction. All of these types of direct extraction are applicable to the current invention.

Solvent Extraction. For practical and economic reasons, hexane is the dominant extraction solvent used for oil extraction from plant material, including plant seeds. However, hexane is very volatile, flammable, and explosive, and consequently, is a physical hazard. In addition, the EPA now categorizes hexane as a hazardous air pollutant. Hexane is included on the list of 189 toxic chemicals, and is controlled under the Toxic Release Inventory of the U.S. EPA Refer. to Inform., Vol. 9, No. 7, July 1998, p. 708.

On the other hand, the use of ethyl acetate for solvent extraction is safe given that it is a natural compound found in some fruits and vegetables. As a result, this process complies with both European and U.S. health laws and requirements.

Further, with respect to the claimed invention, the isothiocyanate, sulforaphane, is not soluble in hexane. Accordingly, pure sulforaphane, spiked into a biphasic mixture of hexane and water, partitions to the aqueous fraction, whereas pure sulforaphane, spiked into a biphasic mixture of ethyl acetate and water, partitions to the organic fraction. Accordingly, a preferred direct solvent extraction employed for the claimed invention is a solvent extraction, using ethyl acetate.

### EXAMPLE 1

### Solvent Extraction Comparison of Broccoli Seed Oil

Sulforaphane was extracted from broccoli seeds of Brassica oleracea cultivar italica, identified from seed lot# DM-1-999A, in separate direct extractions. A solvent extraction using ethyl acetate was compared to one using the “gold standard method” using hexane for seed oil extraction, to provide an oil extractability comparison between the two methods. The analysis of the extractability comparison of ethyl acetate and hexane extraction yielded a similar percentage of oil extracted from the broccoli seeds of seed lot, #DM-1-999-A, as indicated below.

Method for Direct Extraction of Broccoli Seed Oils Using Ethyl Acetate.

Plant extracts were prepared by homogenizing plant tissue in an excess (wt/vol) of ethyl acetate (EtOAc) at room temperature. Seed (20.939 g) was added to 100 ml of EtOAc and homogenized for 3 minutes in a Brinkmann Polytron homogenizer. Homogenates were then centrifuged to separate the oil and aqueous layers. One ml of the supernatant (EtOAc) layer was evaporated in a tared vial and yielded 73 mg of oil. Thus (73 mg oil/ml)/(209.4 mg seed/ml)=about 35% oil.

Method for Direct Extraction of Broccoli Seed Oils Using Hexane.

Plant extracts were prepared by homogenizing plant tissue in an excess (wt/vol) of hexane at room temperature. Seed (20.606 g) was added to 100 ml of Hexane and homogenized for 3 minutes in a Brinkmann Polytron homogenizer. Homogenates were then centrifuged to separate the oil and aqueous layers. One ml of the supernatant (hexane) layer was evaporated in a tared vial and yielded 67 mg of oil. Thus (67 mg oil/ml)/(206.6 mg seed/ml)=about 32% oil.

### EXAMPLE 2

### Sulforaphane Direct Extraction in Broccoli Seed Oil by Ethyl Acetate

A. Preparation of Hydrolyzed Sample.

Seeds (50.301 g) of broccoli (Broccoli oleracea cultivar italica; seed lot# DM-1-999A), were surface-disinfected by immersing in a 25% aqueous solution of Clorox® bleach containing a trace of Alconox® detergent, stirring sporadically for 15 min., and then exhaustively rinsing with sterile water. Glucoraphanin, the precursor of sulforaphane, was the predominant glucosinolate in this seed-lot as determined by HPLC (Troyer et al., J Chromatogr., 919:299-304 (2001)).

Seeds were then strained, and homogenized for 3 minutes in a Brinkmann Polytron homogenizer, in 50 ml of sterile water to which ascorbate had been added to a final concentration of 500 μM. This seed “mash” was then incubated at 37° C. for 3 hours and then overnight at 4° C. Total sample weight was thus (50.301 g seeds +50 g water)=100.3 g.

B. Aqueous Extract of Hydrolyzed Preparation (for Determination of Theoretical Sulforaphane content).

A small sample (0.79 g) of the hydrolyzed seed mash was partitioned into a large excess of water (5 ml) for analytical purposes. This aqueous fraction was centrifuged to remove particulate matter, and injected on High-Performance Liquid Chromatography (HPLC) using an acetonitrile gradient and a C18 column Whatman Partisil ODS-2; (250×4 mm) on a Waters HPLC system equipped with a photodiode array detector tuned to 240 nm., in order to quantify sulforaphane. Quantitation by peak area was in reference to a pure standard. From 0.4 g original seed, 9.15 μmol sulforaphane was recovered, and this turned out to be a low estimate (9.15 μmol/0.4 g≈23 μmol/g seed).

C. Ethyl Acetate Extraction of Hydrolyzed Prep.

The remainder of the hydrolyzed seed mash (100 g-0.79 g) was partitioned into a total of 400 ml EtOAc, by vigorous stirring and shaking. The mixture was then allowed to settle in a large graduate cylinder, the upper 100 ml was removed and evaporated under vacuum. A total mass of 4.108 g was recovered from this 100 ml sample of ethyl acetate extract. Thus, (4.108 g oil/(100/400))/(50.3-0.4) g seed≈33% oil. This oil was then vortexed (1 g volume per 100 ml of water), then allowed to re-equilibrate into two phases, filtered, and the aqueous phase used for HPLC determination of sulforaphane content as described above. It was calculated that 212 mg sulforaphane (1.2 mmol) was present in this aqueous “back-extraction” of the oil respresenting ¼ of the preparation. (Back-extraction is possible, because although the sulforaphane is oil soluble, it also has reasonable solubility in water; thus for analytical purposes (one cannot inject an oil on these HPLC columns), essentially all of the sulforaphane was back-extracted into water for analytical purposes). So 4×212 mg sulforaphane, or 4×1.2 mmol=848 mg sulforaphane (4.8 mmol). Since 4.108 g represents ¼ of the total oil in sample (4×4.108 g of oil=16.43 g oil from 50 g seed); and 4.8 mmol sulforaphane per 16.3 g oil=292 μmol sulforaphane/g broccoli seed oil (or about 98 μmol/g broccoli seed).

The examples described herein are illustrative of the present invention and are not intended to be limitations thereon. Different embodiments of the present invention have been described according to the present invention. Many modifications and variations may be made to the methods and plants described and illustrated herein without departing from the spirit and scope of the invention.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims. All publications and patent applications mentioned in this specification are indicative of the level of skill of those in the art to which the invention pertains.

All publications and patent applications are herein incorporated by reference to same extent as if each individual publication or patent application were specifically and individually indicated to be incorporated by reference in its entirety.

What is claimed is:

1. A method for extracting isothiocyanates from glucosinolate-containing plant material comprising:
a) crushing glucosinolate-containing plant material;
b) moistening the crushed glucosinolate-containing plant material;
c) incubating the moistened and crushed glucosinolate-containing plant material to allow for conversion of glucosinolates to isothiocyanates; and
d) removing oil containing isothiocyanates from the material from part c).

2. The method of claim 1 wherein said plant material is crushed by any of a variety of crushing or grinding or flaking devices common to the seed and grain industry, including but not limited to, dehulling, breaking, grinding, flaking, rolling, pressing, and pelleting.

3. The method of claim 1 wherein said plant material is subjected to thermal treatment to degrade cell walls and to reduce oil viscosity.

4. The method of claim 1 wherein said plant material is moistened with a minimal amount of water, wherein the amount of water needed is adjusted based upon the type of plant material, and is calibrated to moisten, without resulting in an overabundance of standing or free water, in order that the myrosinase enzyme contained within plant tissues can become fully operational.

5. The method of claim 1 wherein said plant material is pre-incubated at a temperature of about 60° C. for the length duration within the range of about 5 minutes to 3 hours to maximize the conversion of glucosinolate to isothiocyanate whilst inactivating the epithiospecifier protein if it exists in that plant material (not all plants contain this protein).

6. The method of claim 1 wherein step c) further comprises the addition of ascorbate, myrosinase, both ascorbate and myrosinase, or any enzyme which facilitates the conversion of isothiocyanate to glucosinolate.

7. The method of claim 1 wherein said oil removal is by a method selected from the group consisting of cold press extraction (screw-press), solvent extraction, fractional distillation, hydraulic pressing, expeller pressing and supercritical CO₂ extraction.

8. The method of claim 6 wherein said extraction with solvents is performed with one or more solvents selected from the group consisting of, but not limited to hexane, methylene chloride, and ethyl acetate.

9. The method of claim 8 wherein said solvent is ethyl acetate.

10. The method of claim 8 wherein said solvent is hexane.

11. The method of claim 1 wherein said isothiocyanate is one or more of sulforaphane, sulforaphene, erysolin, erucin, iberin, alyssin, berteroin, iberverin, cheirolin, 5-methylsulfinylpentyl isothiocyanate, 6-methylsulfinylhexyl isothiocyanate, 7-methylsulfinylheptyl isothiocyanate, 8-methylsulfinyloctyl isothiocyanate (hirsutin), 9-methylsulfinylnonyl isothiocyanate, 10-methylsulfinyldecyl isothiocyanate, phenylethyl isothiocyanate, 4-(α.-L-rhamnopyranosyloxy)benzyl isothiocyanate, 3-(α.-L-rhamnopyranosyloxy)benzyl isothiocyanate, 2-(α L-rhamnopyranosyloxy)benzyl isothiocyanate, 4-(4′-O-acetyl-.alpha.-L-rhamnopyranosyloxy)benzyl isothiocyanate or a derivative thereof.

12. The method of claim 11 wherein said isothiocyanate is sulforaphane.

13. An oil made by the method of claim 1.

14. A pharmaceutical composition comprising the oil of claim 13 and a pharmaceutically acceptable excipient.

15. The pharmaceutical composition of claim 14, wherein said pharmaceutical composition is selected from the group consisting of, but not limited to, antibiotic, antifungal, antihistamine, anti-hypertension, anti-protzoal, antifilarial, anti-malarial, anti-schistosomal, anti-ulcer, anti-coagulant, anti-anxiety, anti-inflammation, antiseptic, nematocidal, antiviral, vasodilators, and protective/prophylactic, and can be administered orally, nasally, parenterally, intrasystemically, intraperitoneally, topically (as by drops of transdermal patch), bucally, or as an oral or nasal spray.

16. The pharmaceutical composition of claim 15 which can be used for human or veterinary applications.

17. A food or drink product, supplement or additive comprising the oil of claim 13.

18. The food or drink product, supplement or additive of claim 17, wherein said food or drink product or supplement is selected from the group consisting ofjuices, smoothies, shakes, teas, soups, sauces, salads, granolas, cereals, breads, other baked goods, fried goods, pills, sprays and other ingestible products.

19. The food or drink product, supplement or additive of claim 18 which can be used for human or veterinary applications.

20. A skin or hair product comprising the oil of claim 13.

21. The skin or hair product of claim 20, wherein said skin or hair product is selected from the group consisting of hair detergents such as shampoo, rinse, rinse-in-shampoo, conditioning shampoo, and the like; various hair cosmetics including hair lotion, hair conditioner, hair treatment, hair cream, hair spray, hair liquid, hair wax, hair water, hair-styling preparation, perming liquid, hair color, acidic hair color, hair manicure, etc.; or various skin cosmetics such as skin lotion, milky lotion, face wash, makeup remover, cleansing lotion, emollient lotion, nourishing cream, emollient cream, massage cream, cleansing cream, body shampoo, hand soap, bar soap, shaving cream, sunburn cosmetics, deodorant gel, deodorant powder, deodorant lotion, deodorant spray, anti-perspirant gel, anti-perspirant powder, anti-perspirant lotion, anti-perspirant spray, combination deodorant & anti-perspirant gel, combination deodorant/anti-perspirant powder, combination deodorant/anti-perspirant lotion, combination deodorant/anti-perspirant spray, makeup removing gel, moisture gel, moisture essence, UV-preventing essence, shaving foam, face powder, foundation, lipstick, cheek rouge, eyeliner, eye shadow, eyebrow pencil, bathing preparation, etc.; mouth detergent such as toothpaste; and other hair and skin products.

22. The skin or hair product of claim of claim 21 which can be used for human or veterinary applications.

23. An agricultural product comprising the oil of claim 13.

24. The agricultural product of claim 23, wherein said agricultural product is selected from the group consisting of agricultural pesticides, powders, pellets, sprays, fertilizers, side-dressings, in-furrow amendments, soil amendments, composts and other agricultural products.

25. A method of inducing the activity of phase 2 enzymes in a mammal comprising administering an effective amount of the pharmaceutical composition of claim 14 to a mammal in need thereof.

26. A method of inducing the activity of phase 2 enzymes in a mammal comprising administering an effective amount of the food or drink product, supplement or additive of claim 17 to a mammal in need thereof.

27. A method of inducing the activity of phase 2 enzymes in a mammal comprising administering an effective amount of the skin or hair product of claim 20 to a mammal in need thereof.

28. A method of inducing the activity of phase 2 enzymes in a plant comprising administering an effective amount of the agricultural product of claim 23 to a plant in need thereof.

29. A method for treating or preventing skin cancer in a mammal comprising administering the skin product of claim 20 to the skin of said mammal.

30. A method for treating or preventing allergic response, arterial occlusion, Alzheimer's Disease, cancer, hypo-cholesterolemia, chronic gastritis, hypertension, joint inflammation (arthritis), macular degeneration, stomach ulcers and gastritis, stroke and upper airway in a mammal comprising administering an effective amount of the pharmaceutical composition of claim 14 to said mammal.

31. A method of making a product containing isothiocyanates comprising:
a) crushing glucosinolate-containing plant material;
b) moistening the crushed glucosinolate-containing plant material;
c) incubating the moistened crushed glucosinolate-containing plant material to allow for conversion of glucosinolates to isothiocyanates;
d) removing oil containing isothiocyanates from the material from part c); and
e) adding the oil from part (d) to a pharmaceutical product, a food or drink product, supplement or additive, a skin or hair product, or an agricultural product.
